(19) United States
(12) Patent Application Publication
(10) Pub. No.: US 2019/0069586 A1
(43) Pub. Date: Mar. 7, 2019

(54) Food Compositions for Weaning

(71) Applicant: Evolve BioSystems, Inc., Davis, CA (US)

(72) Inventors: David KYLE, Davis, CA (US); Samara FREEMAN-SHARKEY, Davis, CA (US); Steven FRESE, Oakland, CA (US)

(21) Appl. No.: 16/084,207

(22) PCT Filed: Mar. 13, 2017
(86) PCT No.: PCT/US17/22207

§ 371 Date: Sep. 11, 2018

Related U.S. Application Data
(60) Provisional application No. 62/307,425, filed on Mar. 11, 2016.

(51) Int. Cl. A23L 33/135; A61K 35/745; A61K 35/747; A23L 33/00; A23L 33/21; A23K 50/30; A23K 10/18; A23K 20/163; A23K 50/60; A23L 11/00; A23L 19/10

(52) U.S. Cl. CPC A23L 33/135; A23Y 2300/25; A61K 35/747; A23L 33/40; A23L 33/21; A23K 50/30; A23K 10/18; A23K 20/163; A23K 50/60; A23L 11/05; A23L 19/105; A23V 2002/00; A23Y 2300/55; A23Y 2300/45; A23Y 2220/71; A23Y 2220/17; A23Y 2300/29; A61K 35/745

(57) ABSTRACT

The inventions described herein relate generally to digestive healthcare, and more particularly, to the feeding of mammals, particularly human infants, who are making a transition from a microbiome with lower diversity to a microbiome with higher diversity. These inventions relate to certain foods comprising a fermentable nutritional component and a probiotic component, where the probiotic component is selected, based on genetic and/or metabolic criteria, to specifically metabolize any Free Sugar Monomers (FSMs) and Free Amino Acids (FAAs) or peptides that accumulate as a result of the fermentable nutritional component in the lower intestine, where they otherwise might be left in the environment to be fermented and metabolized by less adapted/opportunistic bacteria, creating blooms of deleterious intestinal bacteria and shifting the microbiome to a potentially dysbiotic state. The present inventions provide combinations of foods and probiotic bacteria that can protect the mammalian gut from blooms of pathogenic bacteria under the circumstances where the mammalian gut is starting out with a low microbial diversity, such as in weaning infants, or individuals who are post-antibiotic treatment and/or post-chemotherapeutic treatment and transitioning to a higher diversity adapted/stable microbiome.

### FIELD OF THE INVENTION

The inventions described herein relate generally to digestive healthcare, and more particularly, to the feeding of mammals, particularly human infants, who are making a transition from a microbiome with lower diversity to a microbiome with higher diversity. These inventions relate to certain foods comprising a fermentable nutritional component and a probiotic component, where the probiotic component is selected, based on genetic and/or metabolic criteria, to specifically metabolize any Free Sugar Monomers (FSMs) and Free Amino Acids (FAAs) or peptides that accumulate as a result of the fermentable nutritional component in the lower intestine, where they otherwise might be left in the environment to be fermented and metabolized by less adapted/opportunistic bacteria, creating blooms of deleterious intestinal bacteria and shifting the microbiome to a potentially dysbiotic state. The present inventions provide combinations of foods and probiotic bacteria that can protect the mammalian gut from blooms of pathogenic bacteria under the circumstances where the mammalian gut is starting out with a low microbial diversity, such as in weaning infants, or individuals who are post-antibiotic treatment and/or post-chemotherapeutic treatment and transitioning to a higher diversity adapted/stable microbiome.

### BACKGROUND

The intestinal microbiome is the community of microorganisms that live within the gastrointestinal tract, the majority of which is found in the large intestine or colon. In a healthy individual, most dietary carbohydrates that are consumed are absorbed by the body before they reach the colon. Many foods, however, contain indigestible carbohydrates (i.e. dietary fiber) that remain intact and are not absorbed during transit through the gut to the colon. The colonic microbiome is rich in bacterial species that are able to partially consume these fibers and utilize the constituent sugars for energy and metabolism. Methods for measuring dietary fiber in various foods are well known to one of ordinary skill in the art.

In mammalian species, the nursing infant's intestinal microbiome during breast-feeding is quite different from that of an adult microbiome in that the adult gut microbiome generally contains a large diversity of organisms each present as a minor portion of the total population. The nursing infant's microbiome, on the other hand can be made up almost exclusively (up to 80%) of a single species. The transition from the simple, non-diverse microbiome of the nursing infant to a complex, diverse microbiome of an adult correlates with the mammal's transition from a single nutrient source of a rather complex fiber (e.g, mammalian milk oligosaccharides) to more complex nutrient sources that may also have dietary fiber of different composition.

Mammalian milk contains a significant quantity of mammalian milk oligosaccharides (MMO) as dietary fiber. For example, in human milk, the dietary fiber is about 15% of total dry mass. These oligosaccharides comprise sugar residues in a form that is not usable directly as an energy source for the baby or an adult, or for most of the microorganisms in the gut of that baby or adult. Certain microorganisms such as Bifidobacterium longum subsp. infantis (B. infantis) have the unique capability to consume specific mammalian milk oligosaccharides, such as those found in human or bovine milk (see, e.g., U.S. Pat. No. 8,198,872 and U.S. patent application Ser. No. 13/809,556, the disclosures of which are incorporated herein by reference in their entireties). When B. infantis comes in contact with certain MMO, a number of genes are specifically induced; and the products of those genes are responsible for the uptake and internal deconstruction of those MMO. The individual sugar components of these oligosaccharides are then catabolized to provide energy for the growth and reproduction of that organism (Sela et al, 2008).

Mammalian milks evolved to feed two consumers: offspring and their appropriate gut bacteria. The oligosaccharide/glycan portion of the milk is particularly important for the microbiome. If the appropriate bacteria are not present in the body of the mammal, the MMO are not used but are partially or ineffectively degraded, becoming susceptible to non-specific hydrolysis which can thus provide a nutrient source for certain destructive pathogens. The term “mammalian milk oligosaccharide” (MMO), as used herein, refers to those indigestible glycans, sometimes referred to as “dietary fiber”, or the carbohydrate polymers which are not hydrolyzed by the host endogenous enzymes in the digestive tract and remain unabsorbed in the intestinal lumen (e.g., the stomach or small intestine) and reach the large intestine where they may be digested by the microbiome of the mammal. Oligosaccharides may be free in milk or bound to protein or lipids. When bound to protein or lipids, oligosaccharides are referred to as glycans. Oligosaccharides having the chemical structure of the indigestible oligosaccharides found in any mammalian milk or are functionally equivalent are called “MMO” or “mammalian milk oligosaccharides” herein, whether or not they are actually sourced from mammalian milk.

The non-infant mammalian microbiome contains a complexity and diversity of species of bacteria, which develops only after the cessation of milk consumption as a sole source of nutrition. Conventional teaching with regards to the non-infant mammalian microbiome is that complexity provides stability. To be able to effectively consume the complex non-infant diet, maintaining a diversity of microorganisms in the microbiome is thought to be the key to promoting gut health. Lozupone, Nature, Vol. 489, pp. 220-230 (2012).

Treatment of any animal, including all mammals, with antibiotics has an immediate effect of altering the absolute amount and complexity of that animal's microbiome. At the cessation of the course of antibiotic treatment, the rebuilding of the microbiome may be affected by the food being eaten by that mammal and the presence of, or inoculation with, specific bacteria in the intestine. Similar wholesale changes in the microbiome are seen with the use of many chemotherapeutic drugs and therapies, such as fecal microbial transplants.

The transition from a lower diversity unstable, dysbiotic intestinal ecosystem (caused by the use of antibiotics, chemotherapeutic drugs, a change in diet, blooms of pathogenic bacteria, or the like) and the subsequent re-establishment of a complex microbiome of the gastrointestinal tract (GI tract) is a major medical and physiological challenge. This transition often results in the sporadic and damaging proliferation of normally minor bacteria, termed ‘local blooms’ including specific strains of bacteria such as Salmonella, E. coli, Enterobacteria, and Clostridium spp. These blooms of bacteria are in turn detrimental themselves to the host by causing inflammation and direct damage to mucosal cells of the GI tract (Stecher et al 2013). Such bacterial blooms in mammals, depending on the nature of the strains involved, can be reflected in symptoms such as colitis, diarrhea, colic and scours and, under some circumstances, may lead to necrosis, sepsis, and even death.

### SUMMARY OF THE INVENTION

The inventors have discovered that one cause of the bacterial blooms responsible for inflammation and dysbiosis that occur in the microbiome transitional stages is the direct result of the combination of indigestible components (“dietary fiber”) of specific foods consumed by the ‘host’ that reach the large intestine and the pattern by which those food components are broken down by commensal and opportunistic bacteria present in the host's GI tract. Moreover, the inventors have discovered that a critical mechanism underlying these microbial blooms, inflammation, and the resulting dysbiosis, is the breakdown and incomplete absorption of the complex undigested carbohydrates, proteins and peptides by resident colonic bacteria, resulting in the release of large amounts of Free Sugar Monomers (FSMs), Free Amino Acids (FAA's) and small peptides. Access to FSMs, FAA's, and peptides released into the large intestine become an enabling food source which opportunistic bacteria use as a growth substrate. This represents a mismatch between the diet and the bacteria that use those substrates. Not all bacteria in the gut are equal; they have different potentials to use and survive in a complex intestinal nutrient environment. The inventors have discovered that there are better choices for pairing bacteria and dietary fibers during weaning (transitions between new states) to minimize access to any free sugars released as a result of gut activities and any subsequent pathology.

The inventors have further discovered that different FSMs are released from the intact dietary fibers of different food sources in the colon by the action of colonic microbes. These dietary fibers are generally broken down into FSMs by the action of extracellular enzymes produced by various colonic microbes, but these microbes may or may not have the ability to utilize all of the FSMs produced by this enzymatic digestion of the complex oligosaccharides. Indeed, the inventors have discovered that different types of commensal and pathogenic bacteria in the lower GI tract, and particularly in the colon, have different and specific abilities to import and metabolize these FSMs to provide cellular energy. Finally, the inventors have discovered that, by providing specific commensal bacteria as probiotics to an individual who is adding a new source of dietary fiber to their diet, one can minimize the risk of producing blooms of pathogenic microbes that can lead to gut pain, discomfort, or changes in fecal transit times. One mechanism is through controlling the access to FSMs.

This invention provides a composition comprising: (i) a non-milk food, (ii) mammalian milk oligosaccharides (MMO), and (iii) a bacterial culture comprising one or more commensal bacterial species. The bacterial culture is preferably provided in a dose from 10⁷-10¹² cfu. Preferably, the bacterial culture is selected from Lactobacillus, Pediococcus, and/or Bifidobacterium species. The bifidobacteria may be selected from B. longum subsp. longum, B. longum subsp. infantis, B. breve, Bacterium pseudocatanulatum, B. bifidum, B. adolescentis, B. pseudolongum, B. animalis (e.g., B. animalis subsp. animalis, B. animalis subsp. lactis), B. catenulatum, and combinations thereof, the Lactobacillus may be selected from is L. crispatus, L. casei, L. silivarius, L. antri, L. coleohominis, L. pentosus, L. sakei, L. plantarum, and combinations thereof, and Pediococcus may be selected from P. pentosaceus, P. stilesii, P. acidilacti, P. argentenicus, P. claussenii or a combinations thereof.

Non-milk food, if it contains any dietary fiber, does not provide MMO as the majority of the dietary fiber. Infant formula as marketed today would be considered a non-milk food for the purposes of this invention, since it does not include MMO. Preferably, the non-milk food composition contributes a controlled portion of dietary fiber to adapt to the bacterial culture. For example, the non-milk food can contribute about 50%, less than 50%, less than 40%, less than 30%, less than 20%, less than 15%, less than 10%, less than 5%, less than 2.5%, less than 1%, less than 0.5% by weight of the total dietary fiber in the diet, depending on the phase of weaning. In some embodiments, the non-milk food can contribute more than 50%, more than 55%, more than 60%, more than 70%, more than 75%, more than 80%, more than 85%, more than 90%, more than 95%, more than 99% of the dietary fiber by weight in the composition. Fiber is described herein in grams and their percentages are described herein as percent by weight.

Preferably, the milk source of the MMO is from a human, bovine, ovine, equine, or caprine source. Preferably, the MMO contributes a controlled portion of dietary fiber. For example, the MMO can contribute more than 50%, more than 55%, more than 60%, more than 70%, more than 75%, more than 80%, more than 85%, more than 90%, more than 95%, more than 99% by weight of the dietary fiber in the composition, depending on the phase of weaning. In some embodiments, the non-milk food can contribute about 50%, less than 50%, less than 40%, less than 30%, less than 20%, less than 15%, less than 10%, less than 5%, less than 2.5%, less than 1%, less than 0.5% by weight of the dietary fiber in the composition, depending on the phase of weaning.

In one embodiment, the non-milk food can contribute about 50%, less than 50%, less than 40%, less than 30%, less than 20%, less than 15%, less than 10%, less than 5%, less than 2.5%, less than 1%, less than 0.5% by weight of the dietary fiber in the composition, and the MMO can contribute more than 50%, more than 55%, more than 60%, more than 70%, more than 75%, more than 80%, more than 85%, more than 90%, more than 95%, more than 99% by weight of the dietary fiber in the composition. In another embodiment, the non-milk food can contribute more than 50%, more than 55%, more than 60%, more than 70%, more than 75%, more than 80%, more than 85%, more than 90%, more than 95%, more than 99% by weight of the dietary fiber in the composition, and the non-milk food can contribute about 50%, less than 50%, less than 40%, less than 30%, less than 20%, less than 15%, less than 10%, less than 5%, less than 2.5%, less than 1%, less than 0.5% by weight of the dietary fiber in the composition.

In one embodiment, the non-milk food may be a feed for a non-human mammal. The non-human mammal may be a buffalo, camel, rabbit, mouse, rat, pig, cow, goat, sheep, horse, dog, or cat. In some embodiments, the non-human mammal is a laboratory animal. Alternatively, the non-milk food may be a food for a human. In one embodiment, the human may be a baby in need of weaning. The mammal may be on or have just completed a course of oral antibiotics. In one embodiment, the mammal (e.g., human) is on or has just completed a course of chemotherapy, or the mammal (e.g., human) is preparing for, or has just completed, a fecal microbial transplant.

In another embodiment, this invention provides a composition comprising a non-milk baby food and MMO from a human, bovine, equine, or caprine source. Such compositions may be administered for a period to accommodate progressive change in the microbiome, with or without concurrent administration of probiotic bacteria. In a typical embodiment of this invention, the mammal has a microbiome in need of increasing its complexity by at least 10%, preferably by at least 20%, more preferably by at least 30% of the total bacterial species present in the gut. The phrase “increase complexity” when used herein means increasing the complexity based on taxonomic classification of the bacteria in the microbiome of the mammal, and/or increasing the complexity based on the proportional number of bacteria by classification in the microbiome of the mammal, which may be generally calculated from the amount of DNA with sequences specific to a particular genus, species, or strain normalized against the total amount of DNA sequences in stool.

In one embodiment, gut microbiome complexity of a mammal (e.g., a human infant) is increased by providing a dietary composition comprising a non-milk food, MMO, and a bacterial culture, where the MMO are from human, bovine, ovine, equine or caprine milk or the MMO are non-milk oligosaccharides substantially identical to human milk oligosaccharides, bovine milk oligosaccharides (BMO), caprine milk oligosaccharides (CMO), porcine milk oligosaccharides (PMO), equine milk oligosaccharides (EMO), and/or ovine milk oligosaccharides (OMO). The bacterial culture can be chosen from Bifidobacterium, Pediococcus, and Lactobacillus, which may be provided in a daily dose of from 10⁷-10¹² cfu.

In another mode, this invention provides methods of increasing the gut microbiome complexity in a mammal by administering any of the compositions described herein to the mammal. In one embodiment, gut microbiome complexity of a mammal (e.g., a human infant) is increased by providing a dietary composition comprising a non-milk food and a bacterial culture, to the mammal (e.g., a human infant), where the mammal is contemporaneously receiving MMO from another source (e.g., mother's milk). The bacterial culture can be chosen from Bifidobacterium, Pediococcus, and Lactobacillus, which may be provided in a daily dose of from 10⁷-10¹² cfu.

In another embodiment, gut microbiome complexity is increased in a mammal during or following antibiotic therapy by providing a dietary composition comprising a non-milk food, MMO, and a bacterial culture, where the MMO are from human, bovine, ovine, equine, or caprine milk or the MMO are non-milk oligosaccharides substantially identical to HMO, BMO, CMO, EMO, and/or OMO, and the bacterial culture provides probiotic bacteria chosen from Bifidobacterium, Pediococcus, and Lactobacillus, or combinations thereof. The bacterial culture may be provided in a daily dose of from 10⁷-10¹² cfu. In any of these embodiments, at least one of the bacterial species is preferably Bifidobacteria longum subsp. infantis.

Typically, feeding the controlled diet to the mammal is continued for a period of days to weeks, for example, following the reduction in breastmilk, an increase in formula feeding, an increase in complementary foods, administration (and/or cessation) of antibiotics, administration (and/or cessation) of chemotherapy, and infusion of the fecal microbial transplant composition. Any of the embodiments described herein may include the administration of compositions of varying MMO and non-milk food dietary fibers. For example, the initial stage of administration may include a composition where the MMO provides more than 50% of the dietary fiber of the composition, and where the non-milk food provides less than 50% of the dietary fiber of the composition. A later stage of administration may include a composition where the MMO provides less than 50% of the dietary fiber of the composition, and where the non-milk food provides more than 50% of the dietary fiber of the composition.

In still another mode, this invention provides a method of increasing the gut microbiome complexity in a human in need thereof by: (a) initiating a controlled diet comprising low fiber food and 5-40 g/day of MMO for said human for from 2-7 days prior to a fecal microbial transplant (FMT); (b) preparing a modified FMT composition comprising the fecal microbiome from a healthy individual, at least 5 g/day of MMO, and from 1-1000×10⁸ cfu of Bifidobacterium longum subsp. infantis; (c) infusing the colon of said human with the modified FMT composition; and (d) following the FMT with the controlled diet of step (a) for from 0 to 7 days. Preferably, the MMO comprise from at least from 20% to at least 70% of the total dietary oligosaccharides of the controlled diet.

In yet another mode, this invention provides a method of increasing the gut microbiome complexity in a human in need thereof consisting of (a) preparing a dry composition of a weaning food by cooking the food, drying the cooked food and milling the dried food to a powder usable as a weaning food; (b) growing a culture of Bifidobacterium and/or Lactobacillus which is selected from a group that consumes FSMs found in the feces of an infant fed a similar weaning food, harvesting the culture and drying the cell mass in the presence of a preservative; and (c) combining the dry composition of weaning food with the dry composition of bacterial culture in a ratio of from 10⁸-10¹² cfu of bacterial culture to 100 g of weaning food.

### BRIEF DESCRIPTION OF THE DRAWINGS

FIG. 1: Chart showing the base 10 log change in B. infantis levels by day during mucosal healing diet including BMO, GOS, and B. infantis. The data are reported as CFU B. infantis per ug DNA divided by CFU total bacteria per ug DNA.

FIG. 2A: A plot showing the addition of B. infantis to a breast-fed infant. B. infantis was provided contemporaneously with breast milk to establish the dominance of B. infantis in the infant gut. The B. infantis levels were maintained by keeping the majority of fiber coming from human milk. Complementary foods were introduced at low levels during this time period.

FIG. 2B: A plot showing the addition of B. infantis to a breast-fed infant. B. infantis was provided contemporaneously with breast milk to establish the dominance of B. infantis in the infant gut. The B. infantis levels were maintained by keeping the majority of fiber coming from human milk. Complementary foods were introduced at low levels during this time period.

FIG. 3A: A plot showing the same introduction of B. infantis to the infant as in FIGS. 2A-B. B. infantis was provided contemporaneously with breast milk to establish the dominance of B. infantis in the infant gut. However, this infant switched their diet to a non-milk food, infant formula with and without low-level complementary feeding. The overall effect was a net reduction in MMO and a decrease in the abundance of B. infantis at later time points.

FIG. 3B: A plot showing the same introduction of B. infantis to the infant as in FIGS. 2A-B. B. infantis was provided contemporaneously with breast milk to establish the dominance of B. infantis in the infant gut. However, this infant switched their diet to a non-milk food, infant formula with and without low-level complementary feeding. The overall effect was a net reduction in MMO and a decrease in the abundance of B. infantis at later time points.

### DETAILED DESCRIPTION OF THE INVENTION

Certain embodiments of the instant invention pertain to food and probiotic compositions, formulated and used for the express purpose of increasing the diversity of the microbiota in the colon, where such uses include, but are not limited to, the weaning of an infant mammal from its mother's milk, the weaning of any mammal from a course of antibiotics, the weaning of any mammal from a medical procedure that reduces microbiome complexity (e.g., a course of chemotherapy, or use of total enteral nutrition), or the preparation for and application of a FMT procedure to increase microbiome complexity. In a preferred embodiment, the mammal includes, but is not limited to, a human, pig, cow, goat, sheep, horse, dog, or cat. In a particularly preferred embodiment, the mammal is a human. In some embodiments of the invention, the probiotic bacteria include, but are not limited to, commensal bacteria that typically reside in the lower intestine, or colon. In a preferred embodiment, the bacteria include, but not limited to, those of the genus Lactobacillus, Pediococcus and Bifidobacterium. In certain embodiments of the instant invention, the foods include, but are not limited to, complex oligosaccharides and glycans from meat, fish, milk, eggs, shellfish, fruits, vegetables, grains, nuts, and seeds in whole or a processed form. In some embodiments of the instant invention, certain bacterial species including, but not limited to, those from the genus Lactobacillus, Pedicococcus or Bifidobacterium, are combined and delivered with the food in a way that facilitates consumption of FSMs in the GI tract by commensal bacteria, which mitigates the possibility of pathogenic blooms of unwanted or unhealthy bacteria. See, e.g., International Publication No. WO 2016/149149, the disclosure of which is incorporated herein by reference in its entirety.

A simple, healthy microbiome can be described as the presence of greater than 10⁸ cfu/g stool of a single genus of bacteria (e.g., Bifidobacterium), more particularly, of a single species or strain of bacteria (e.g., B. longum subsp. infantis [B. infantis]). For example, up to 80% of the microbiome can be dominated by the bacteria or, more particularly, by the single subspecies of a bacteria. A simple microbiome can also be described as the presence of greater than 20%, 30%, 40%, 50%, 60%, 70%, 75%, 80%, or 90% of a single genus of bacteria (e.g., Bifidobacterium), more particularly, of a single subspecies of bacteria (e.g., B. longum subsp. infantis [B. infantis]). Increasing complexity of the microbiome can be described as decreasing the presence of the dominating genus of bacteria (e.g., Bifidobacterium) or subspecies of bacteria (e.g., B. longum subsp. infantis [B. infantis]) by at least 5%, at least 10%, at least 15%, at least 20%, at least 25%, at least 30%, at least 35%, at least 40%, at least 45%, at least 50%, at least 55%, at least 60%, at least 65%, at least 70%, or at least 80% in the microbiome. A decrease in the presence of the dominating genus of bacteria (e.g., Bifidobacterium) or subspecies of bacteria (e.g., B. longum subsp. infantis [B. infantis] in a human infant) allows for the reintroduction of a diversity of bacteria genera and species into the microbiome.

A patient having a “simpler microbiome” or “less diverse microbiome” can be described as a patient that has 10⁸ cfu/g stool or greater levels of one particular species or one strain of microorganism in the gut, for example, at least 10⁹ cfu/g stool, at least 10¹⁰ cfu/g stool, or at least 10¹¹ cfu/g stool. A simple microbiome can also be described as the presence of greater than 20%, 30%, 40%, 50%, 60%, 70%, 75%, 80%, or 90% of a single genus of bacteria (e.g., Bifidobacterium), more particularly, of a single subspecies of bacteria (e.g., B. longum subsp. infantis [B. infantis]). A simple microbiome may be healthy in the case of an infant whose diet is almost entirely composed of a single nutrient source (e.g., mother's milk). However, for an individual consuming a more varied diet, a shift of the microbiome to simpler structure is typically an indication of dysbiosis. This includes patients with a bacterial bloom that rapidly expands the presence of a particular organism, or patients with reduced diversity where key commensal species are missing. Both of these cases may present as a microbiome less diverse than expected in a healthy individual, and these patients are characterized as having a dysbiotic microbiome. Shifts in the microbiome can be determined using Next Generation Sequencing (see, e.g., Ji et al., “From next-generation sequencing to systematic modeling of the gut microbiome”, Front Genet. (Jun. 23, 2015), published online at doi.org/10.3389/fgene.2015.00219) or full Metagenomics (see, e.g., Wang et al., “Application of metagenomics in the human gut microbiome”, World J. Gastroenterol. (2015), Vol. 21, No. 3, pp. 803-814) approaches to monitor the change in specific organisms, or overall shifts in families known to contain members of opportunistic or pathogenic organisms. Typically, measurements can be normalized using the amount of DNA per gram of stool.

Mammalian milk contain a significant quantity of mammalian milk oligosaccharides (designated herein as “MMOs”) in a form that is not usable as an energy source for the milk-fed mammal. MMOs are also not digestible by most of the microorganisms in the gut of that mammal. MMOs can be found as free oligosaccharides (soluble fiber) or conjugated to protein or lipids (“dietary glycans”). The term “mammalian milk oligosaccharide”, as used herein, includes those indigestible oligosaccharides and glycans, sometimes referred to as “dietary fiber”, or the carbohydrate polymers which are not hydrolyzed by the endogenous enzymes in the digestive tract (e.g., the small intestine) of the mammal. Oligosaccharides having the chemical structure of the indigestible oligosaccharides found in any mammalian milk are collectively called “MMO” or “mammalian milk oligosaccharides” herein, whether or not they are actually sourced from mammalian milk. For human milk oligosaccharides (“HMOs”), the major HMOs in milk include lacto-N-tetraose (LNT), lacto-N-neotetraose (LNnT) and lacto-N-hexaose, which are neutral HMOs, in addition to fucosylated oligosaccharides such as 2-fucosyllactose (2FL), 3-fucosyllactose (3FL), difucosyllactose, and lacto-N-fucopentaoses I, II, III, and V. Acidic HMOs include sialyl-lacto-N-tetraose, 3′ and 6′ sialyllactose (6SL), and 3′-sialyllactosamine, 6′-sialyllactosamine, and 3′-sialyl-3-fucosyllactose. HMOs are particularly highly enriched in fucosylated oligosaccharides (Mills et al., U.S. Pat. No. 8,197,872). Among the enzymes that produce HMOs in the mammary gland is the enzyme encoded by the 2-fucosyltransferase (FUT2) gene, which catalyzes the linking of fucose residues by an α1,2-linkage to oligosaccharides found in human milk. Fucosylated oligosaccharides are known to inhibit the binding of pathogenic bacteria in the gut. HMOs, and in particular the fucosylated HMOs, share common structural motifs with glycans on the infant's intestinal epithelia known to be receptors for pathogens. (German et al., WO 2012/009315).

While a human infant is consuming human breast milk as the sole source of nutrition, it has a gut microbiome that is dominated by a single bacterial species—Bifidobacteria longum subsp. infantis. The nutritional energy source of this organism is primarily the human milk oligosaccharides (HMOs) that represent a significant fraction of breast milk (approximately 15%), and this organism, in turn, provides a number of benefits to the growing and developing baby (Underwood et al¹). These HMOs and their use have been previously described (U.S. Pat. No. 8,197,872, the disclosure of which is incorporated herein by reference in its entirety). The inventors have also discovered that other MMOs can also be used as carbon sources by certain bifidobacteria including, but not limited to, B. breve, B. pseudocatanulatum and/or B. longum (described in detail in U.S. Pat. No. 9,200,091 and PCT/US2015/057226, the disclosures of which are incorporated herein in their entireties). ¹ Underwood, M A, J B German, C B Lebrilla, and D A Mills (2015). Bifidobacterium longum subsp. infantis: champion colonizer of the human gut. Pediatr Res, 77: 229-235

A mammal that is receiving a sole source of nutrition (e.g., oligosaccharides of the sort found in mammalian milk (MMO)), such as, but not limited to a breast fed human infant, and where the microbiome of this mammal is dominated by one or a few species of microbe that are particularly adapted to grow on those oligosaccharides as a carbon source, can be weaned to a more complex and varied diet by administering a diet comprising the following: a bifidobacteria (e.g., B. infantis), MMO in a reduced amount, non-milk oligosaccharide compound(s) of the new dietary component(s) in an amount less than that of the MMO, and a probiotic composition competent to metabolize the sugar components of the non-milk oligosaccharides. After a period on this diet, the diet administered to this mammal is adjusted by reducing the amount of the bifidobacteria (e.g., B. infantis) while MMO and the amount of non-milk oligosaccharide compound(s) is increased along with additional probiotic cells. Successive stages of continued decreasing and/or increasing, respectively, of the components can follow. In any of the above embodiments, the probiotic composition should be selected based on the non-milk oligosaccharide compound. For example, the probiotic composition can be selected based on the carbohydrate residues present in the non-milk oligosaccharide compound(s), and the bacteria's preference for the carbon compound(s).

In typical embodiments of the instant invention, the non-milk oligosaccharide compound(s) can be included in a food composition. The food composition can comprise non-milk nutritional components for an infant mammal including, but not limited to, applesauce, avocado, banana, squash, carrots, green beans, oatmeal, peaches, pears, peas, potatoes, cereal, sweet potatoes, meat, and fish in natural or pureed form, alone or in combination with each other, and MMO. In a preferred embodiment of the invention, the MMO includes, but is not limited to, a human milk oligosaccharide (HMO), a bovine milk oligosaccharide (BMO), a bovine colostrum oligosaccharide (BCO), and a goat milk oligosaccharide (GMO), or any single purified MMO or any combination thereof. Preparation methods for such compositions are described, for example, in U.S. Pat. Nos. 8,197,872 and 9,200,091, and International Publication No. WO 2016/065324, the disclosures of which are incorporated herein by reference in their entirety. In typical embodiments, the MMO of the food composition is present in an amount of from about 10 to 5,000 mg/oz of food. In a more preferred embodiment the MMO is present in an amount of from 50-1,000 mg/oz of food. In a particularly preferred embodiment, the MMO is present in an amount of from 100-500 mg/oz of food. In an alternative embodiment, the MMO may comprise dietary or soluble fiber oligosaccharides from milk of more than one species of mammal or can be produced from sources other than milk. In another preferred embodiment, the MMO may be substituted by oligosaccharides from sources other than milk, including but not limited to MMO produced by recombinant bacterial or chemical processes and/or galactooligosaccharide (GOS) preparations that provide selective growth of certain bifidobacteria such as B. longum subsp, infantis and B. breve as described in U.S. Pat. No. 8,425,930, the contents of which is incorporated herein by reference.

In other embodiments of the invention, the food composition comprises nutritional components for a mammal, MMO, and a bifidobacteria including, but not limited to, B. breve, B. pseudocatanulatum, B. longum, B. adolescentis, B. pseudolongum, and B. animalis. In a more preferred embodiment, the Bifidobacterium of the composition is Bifidobacterium longum subspecies infantis. In typical embodiments, the Bifidobacterium is provided in an amount of from 10⁶-10¹¹ cfu/serving of food wherein one serving represents 20% of the total daily recommended allocation of calories for a mammal (e.g., an infant) on the basis of size and weight. In a more preferred embodiment, the Bifidobacterium is provided in an amount of from 10⁷-10¹⁰ cfu/oz. of food (e.g., baby food). In a particularly preferred embodiment, the Bifidobacterium is provided in an amount of from 10⁸-10⁹ cfu/oz. of food (e.g., baby food).

In some embodiments of the instant invention, the food composition comprising the nutritional components for the mammal and the MMO are premixed and loaded into a container (e.g., a squeezable pouch) made from material including, but not limited to, polyester, aluminum, and/or polyethylene, or combinations thereof. In one embodiment, the food composition is a baby food composition, and the container is a squeezable pouch. The bifidobacteria may be dry-coated on the inside of the spout such that the bifidobacteria is not in contact with the baby food until the baby food is squeezed from the tube. In other embodiments of the invention, the bifidobacteria is provided in a sachet that is opened and mixed with the food/MMO composition immediately before consumption (e.g., feeding to an infant).

Some embodiments of the invention relate to a method to maintain or provide at least about 10%, at least about 20%, at least about 30%, at least about 40%, or at least about 50% of an infant mammal's microbiome as Bifidobacterium (e.g., B. infantis) during at least a portion of the weaning process by providing a weaning food comprising a food source appropriate for an infant mammal, MMO, and bifidobacteria (e.g., B. infantis).

Some embodiments of the invention relate to a method to facilitate the recovery of the GI tract from a treatment with antibiotics by restoring the gut microflora first with a microbiome similar to that of a breast-fed baby (i.e., a simple microbiome dominated by bifidobacteria). This method involves putting the patient on a daily dietary regimen wherein the dietary fiber from MMO such as, but not limited to, HMO, BMO, BCO, GMO, GOS single purified MMO therefrom, or combinations thereof, constitutes at least 20%, at least 30%, at least 40%, at least 50%, at least 60%, at least 70%, at least 80%, or at least 90% of the total fiber glycan (oligosaccharide) consumed on a daily basis by that individual. In an alternative embodiment, the MMO may comprise dietary fiber oligosaccharides from milk of more than one species of mammal. The daily dietary regimen can also contain a daily dose of bifidobacteria including, but not limited to, B. breve, B. pseudocatanulatum, B. longum, B. adolescentis, B. pseudolongum, and B. animalis. In a more preferred embodiment, the bifidobacteria of the composition is Bifidobacterium longum subspecies infantis. In one embodiment, the bifidobacteria is provided in an amount of from 10⁷-10¹² cfu/day, from 10⁸-10¹¹ cfu/day, or from 10⁹-10¹⁰ cfu/day.

In one embodiment, the daily dietary regimen continues for from 1-30 days, for example. The daily dietary regimen can contain different stages of administration. For example, at stage 1, the daily dietary regimen can contain a composition where the dietary fiber from MMO constitutes at least 50%, at least 60%, at least 70%, at least 80%, or at least 90% of the total fiber glycan consumed on a daily basis by that individual. At stage 2, the daily dietary regimen can contain a composition where the dietary fiber from MMO constitutes at least 50%, at least 40%, at least 30%, at least 20%, at least 10%, or at least 5%, of the total fiber glycan consumed on a daily basis by that individual.

Some embodiments of the invention include a composition and a method to facilitate the recovery of the complexity of the GI tract generated by a FMT. This method involves starting the patient on a daily dietary regimen from about 2 to about 7 days prior to a fecal microbial transplant wherein the dietary regimen comprises dietary fiber from MMO such as, but not limited to, HMO, BMO, BCO, GMO, GOS, single purified MMO therefrom, or combinations thereof, and further constitutes at least 20%, at least 30%, at least 40%, at least 50%, at least 60%, at least 70%, at least 80%, at least 90%, or essentially 100% of the total daily dietary fiber consumed by the patient on a daily basis. In some embodiments of the instant invention, the FMT itself is supplemented with from 1 to 20 g of MMO such as, but not limited to, HMO, BMO, BCO, GMO, GOS, single purified MMO therefrom, or combinations thereof, and a bifidobacteria including, but not limited to, B. breve, B. pseudocatanulatum, B. longum, B. adolescentis, B. pseudolongum, and B. animalis prior to inoculation of the patient with the FMT. In a more preferred embodiment the Bifidobacterium of the composition is Bifidobacterium longum subspecies infantis. In one embodiment, the Bifidobacterium is provided in an amount of from 10⁷-10¹² cfu, from 10⁸-10¹¹ cfu, or from 10⁹-10¹⁰ cfu.

In another embodiment, the invention includes a method for the explicit measurement of intestinal FSM's and FAA's and free peptides (FP) for the selection of probiotic bacteria whose addition to the diet directly, or as supplements, achieves the consumption of FSMs and FAAs. Consumption of FSMs and FAAs and FPs may be demonstrated by the reduction or absence of FSMs and FAA's in the feces of the mammal or a shift in specific clades away from enterobacteracieae and proteobacteria and detection of the species added below. For example, if the non-milk food being included in the mammal's diet is rice, which has an expected FSM that is left over after the normal digestive process of glucose (see Table 1, describing expected FSMs for various non-milk foods), one could select a probiotic bacteria that prefers to consume glucose (see Table 2, describing preferred FSM consumption by various bacteria) (e.g., B. longum, B infantis, B. pseudocatanulatum, B. bifidum, B. breve, B. adolescentis, B. pseudolongum, B. animalis, L. plantarum, L. casei, L. rhamnosus (e.g., LGG), L. acidophilus, L. curvatus, L. reuteri, L. brevis, L. fermentum, L. crispatus, L. johnsonii, L. gasseri, L. mucosae, and L. salivarius, P. pentosaceus, P. stilesii, P. acidilacti, P. argentenicus, P. claussenii).

Some embodiments of the invention include a composition comprising Pediococcus, Lactobacillus and/or Bifidobacterium and a non-milk specific dietary fiber from a food source appropriate for mammals (e.g., humans). In one embodiment, such compositions can be delivered to the subject in need thereof in the form of a food including, but not limited to, a baby food, a weaning food, enteral nutrition, and a medical food to be consumed by a mammal (e.g., human) of any age. In another embodiment, such compositions can also be delivered to the subject in need thereof in the form of a powder intended to be mixed with water or a nutritive liquid, pudding, or gel. In yet another embodiment, such compositions can be delivered to the subject in need thereof in the form of a tablet, capsule, enema, or suppository. In some embodiments of the invention, the composition additionally includes a MMO including, but not limited to, a glycan from HMO, BMO, BCO, GMO, or GOS, as an individual oligosaccharide or glycan, or a combination of oligosaccharides or glycans, and the MMO is present in an amount from about 10 mg/oz. of food to 5,000 mg/oz. of food. In a more preferred embodiment, the glycan is present in an amount of from 50 mg/oz of food to 2,000 mg/oz of food. In one embodiment, the milk glycan is present in an amount of from 100 mg/oz of food to 500 mg/oz of food.

A number of weaning foods are provided in Table 1, showing where the inventors have determined the most likely FSMs that are released by the partial digestive degradation of their component oligosaccharides. Such FSMs include, but are not limited to, sialic acid, fucose, rhamnose, mannose, glucose, gluconate, glucuronic acid, galacturonic acid, arabinose, fructose, xylose, N-acetyl glucosamine, N-acetylgalactosamine, and N-glycoyl-neuraminic acid.

A number of bacterial species are provided in Table 2, since the inventors have discovered preferred carbon source(s) for certain bacteria. Certain embodiments of the invention would include one or more of any of the species found in Table 2.

In some embodiments, a powdered composition of Bifidobacterium is prepared by fermentation using processes known in the art, such as Kiviharju et al². In one embodiment, a powdered composition of Bifidobacterium is prepared by activation processes (e.g., as described in PCT/US2015/057226, the contents of which is incorporated herein in its entirety). The final dried powder is diluted with an excipient such as, but not limited to, lactose, cellulose, hydroxymethylcellulose, silica, a milk glycan, and magnesium stearate, to a concentration of from 10⁷-10¹² cfu/g, preferably from 10⁸-10¹¹ cfu/g, and more preferably from 10⁹-10¹⁰ cfu/g and added to a food product that is not primarily a milk product. In a preferred embodiment, the Bifidobacterium is B. longum, B. pseudocatanulatum, B. bifidum, B. breve, B. adolescentis, B. pseudolongum, B. animalis. The composition may also comprise MMO including, but not limited to an oligosaccharide from HMO, BMO, BCO, GMO, and/or GOS as an individual MMO, or a combination of MMOs. The food product may also include, but is not limited to, a baby food, a weaning food, enteral nutrition, and a medical food to be consumed by a mammal (e.g., a human) of any age. In some preferred embodiments, the composition of Bifidobacterium, the food product, and the MMO are formulated and used for the express purpose of increasing the diversity of the microbiota in the colon, where such increases include, but are not limited to, the weaning of an infant mammal from its mother's milk, the weaning of any mammal from a course of antibiotics, the weaning of any mammal from a medical procedure that reduces microbiomal complexity (e.g., a course of chemotherapy, gastric bypass, or use of total enteral nutrition), or the application of a FMT procedure to increase microbiomal complexity. Another aspect of the invention is the use the composition to reduce or eliminate the production of blooms of pathogenic microbes that can lead to gut pain, discomfort, or changes in fecal transit times. ² Kiviharju K, Leisola M, and Eerikainen T. (2015) Optimization of a Bifidobacterium longum production process. J Biotechnol. 2005 May 25; 117(3):299-308.

In some embodiments, a powdered composition of Lactobacillus is prepared by feimentation using processes known in the art, such as Chang et al³, and the final dried powder is diluted with an excipient such as, but not limited to, lactose, cellulose, hydroxymethylcellulose, silica, a milk oligosaccharide, and magnesium stearate, to a concentration of from 10⁷-10¹² cfu/g, preferably from 10⁸-10¹¹ cfu/g, and more preferably from 10⁹-10¹⁰ cfu/g. In a preferred embodiment, the Lactobacillus is L. plantarum, L. casei, L. rhamnosus (e.g., LGG), L. acidophilus, L. curvatus, L. reuteri, L. brevis, L. fermentum, L. crispatus, L. johnsonii, L. gasseri, L. mucosae, and/or L. salivarius. ³ Chung et al, “Cultivation of Lactobacillus crispatus KLB46 Isolated from Human Vagina,”Biotechnol. Bioprocess Eng. (2001), Vol. 6, pp. 128-132.

In some embodiments, the probiotic composition also comprises MMO including, but not limited to, MMO from HMO, BMO, BCO, GMO, or GOS as an individual MMO, or a combination or mixture of MMOs. Preferred embodiments provide a food product which includes, but is not limited to, a baby food, a weaning food, enteral nutrition, and a medical food to be consumed by a mammal (e.g., a human) of any age. In some embodiments, the composition of the Lactobacillus, the food product, and the MMO is formulated and used for the express purpose of increasing the diversity of the microbiota in the colon wherein such increases include, but are not limited to, the weaning of an infant mammal from its mother's milk, the weaning of any mammal from a course of antibiotics, the weaning of any mammal from a medical procedure that reduces microbiomal complexity (e.g., a course of chemotherapy, or use of total enteral nutrition), or the application of a FMT procedure to increase microbiomal complexity. Another aspect of the invention is to use the use the composition to eliminate the production of blooms of pathogenic microbes that can lead to gut pain, discomfort, or changes in fecal transit times.

Some embodiments of the instant invention include a probiotic composition comprising bifidobacteria lactobacillii. Preferably, the Bifidobacterium is B. longum and Lactobacillus, L. crispatus where the B. longum is present at from 10⁷-10¹² cfu from 10⁸-10¹¹ cfu, or from 10⁹-10¹⁰ cfu, and L. crispatus is present at from 107-10¹² cfu, from 10⁸-10¹¹ cfu, or from 10⁹-10¹⁰ cfu, as a daily dose in a food source. In some embodiments, the food source of the composition further comprises a vegetable fiber and/or a MMO. In other embodiments, the composition of the Bifidobacterium, and Lactobacillus, the food product, and the MMO is formulated and used for the express purpose of increasing the diversity of the microbiota in the colon wherein such increases include, but are not limited to, the weaning of an infant mammal from its mother's milk, the weaning of any mammal from a course of antibiotics, the weaning of any mammal from a medical procedure that reduces microbiomal complexity (e.g., a course of chemotherapy, gastric bypass, or use of total enteral nutrition), or the application of a FMT procedure to increase microbiomal complexity. Another aspect of the invention is to use the use the composition to suppress or eliminate the production of blooms of pathogenic microbes that can lead to gut pain, discomfort, or changes in fecal transit times. In some embodiments, the Bifidobacterium is activated.

Some embodiments of the instant invention include a probiotic composition comprising B. bifidum and/or a B. longum and L. casei wherein bifidobacteria is present at from 10⁷-10¹² cfu, from 10⁸-10¹¹ cfu/g, or from 10⁹-10¹⁰ cfu and L. casei is present at from 10⁷-10¹² cfu, from 10⁸-10¹¹ cfu, or from 10⁹-10¹⁰ cfu as a daily dose in a food source. In another embodiment, the food source of the composition further comprises a cereal fiber and/or a MMO. In another embodiment, the food source of the composition further comprises a vegetable fiber and/or a MMO. In another embodiment, the composition of the Bifidobacterium and Lactobacillus, the food product, and the MMO is formulated and used for the express purpose of increasing the diversity of the microbiota in the colon wherein such increases include, but are not limited to, the weaning of an infant mammal from its mother's milk, the weaning of any mammal from a course of antibiotics, the weaning of any mammal from a medical procedure that reduces microbiomal complexity (e.g., a course of chemotherapy, or use of total enteral nutrition), or the application of a FMT procedure to increase microbiomal complexity. Another aspect of the invention is to use the composition to eliminate the production of blooms of pathogenic microbes that can lead to gut pain, discomfort, or changes in fecal transit times. In some embodiments, the Bifidobacterium is activated.

Some embodiments of the instant invention include a probiotic composition comprising B. breve and L. plantarum wherein the B. breve is present at from 10⁷-10¹² cfu/g, from 10⁸-10¹¹ cfu/g, or from 10⁹-10¹⁰ cfu/g, and L. plantarum is present at from 10⁷-10¹² cfu/g, from 10⁸-10¹¹ cfu/g, or from 10⁹-10¹⁰ cfu/g, as a daily dose in a food source. In another embodiment, the food source of the composition further comprises a meat or fish fiber and/or a MMO. In another embodiment, the food source of the composition further comprises a vegetable fiber and/or a MMO. In another embodiment, the composition of the Bifidobacterium and Lactobacillus, the food product, and the MMO is formulated and used for the express purpose of increasing the diversity of the microbiota in the colon wherein such increases include, but are not limited to, the weaning of an infant mammal from its mother's milk, the weaning of any mammal from a course of antibiotics, the weaning of any mammal from a medical procedure that reduces microbiomal complexity (e.g., a course of chemotherapy, or use of total enteral nutrition), or the application of a FMT procedure to increase microbiomal complexity. Another aspect of the invention is to use the composition to eliminate the production of blooms of pathogenic microbes that can lead to gut pain, discomfort, or changes in fecal transit times. In some embodiments, the Bifidobacterium is activated.

Certain embodiments of the present invention involve the delivery of a food source to a mammal as the means of weaning from its mother's milk (a “weaning food”) or in order to recover from an antibiotic treatment, use of a chemotherapeutic agent or a fecal transplant (a “recovery food”), and one of more species of bacteria selected to consume the FSMs that would be released from that food source. Certain embodiments of the present invention involve the staged addition of a weaning food or a recovery food to infants or other mammals in need of weaning or recovery, that progressively increase the complexity of dietary fiber and complementary probiotic supplements to prevent the production of excess FSM's in the colon resulting in a non-commensal bacterial overgrowth that leads to gut pain, discomfort, or changes in fecal transit times.

In some embodiments, the weaning occurs in stages. In some embodiments, the weaning occurs by successively increasing the proportion of dietary fiber from a non-milk source (e.g., MMO). The weaning process can occur in one, two, three, or more stages. For example, the weaning process includes a first stage, where the composition includes bifidobacteria (e.g., B. infantis), a non-milk food, and, optionally, MMO. The composition in the first stage would include non-milk food that contributes 10% or less of the dietary fiber of the mammal's total daily dietary fiber. If the mammal is an infant that is being weaned while being breast-fed, the mammal may be provided a composition that includes a non-milk food and bifidobacteria, while the mammal is receiving MMOs from another source (e.g., mother's milk). In some embodiments, the composition can also include MMOs in an amount equal to 90-100% of that found in the diet of an exclusively breast-fed infant. In some embodiments, the composition can include MMO in an amount necessary to allow for the total amount of MMO in the mammal to be equal to 90-100% of that found in the diet of an exclusively breast-fed infant.

The first stage of the weaning process can be administered for a period of from one day to six months. For example, the first stage can be administered for one day, two days, three days, four days, five days, six days, seven days, eight days, nine days, ten days, eleven days, twelve days, thirteen days, fourteen days, fifteen days, eighteen days, three weeks, four weeks, five weeks, six weeks, seven weeks, eight weeks, nine weeks, ten weeks, eleven weeks, twelve weeks, thirteen weeks, fourteen weeks, fifteen weeks, four months, five months, or six months.

The second stage of the weaning process may include a composition with comparatively increased amounts of dietary fiber coming from non-milk food. For example, the non-milk food can contribute 10% or more (e.g, between 10% and 50%) of the dietary fiber of the mammal's total daily dietary fiber. If the mammal is an infant that is being weaned while being breast-fed, the mammal may be provided a composition that includes a non-milk food and bifidobacteria, while the mammal is receiving MMOs from another source (e.g., mother's milk). In some embodiments, the composition may include MMO in an amount that is equal to 50-89% of that found in the diet of an exclusively breast-fed infant. In some embodiments, the composition can include MMO in an amount necessary to allow for the total amount of MMO in the mammal to be equal to 50-89% of that found in the diet of an exclusively breast-fed infant.

In some embodiments, the bacterial culture is selected based on the non-milk food. For example, the bacterial culture can be selected based on the FSMs released by the non-milk food (see, Table 1) and the bacteria's preferred consumption of these FSMs (see Table 2). This is creating the environment where the new fibers are used successfully and does not leave room for pathogenic blooms while other organisms take a more prominent place in the microbiome.

The second stage of the weaning process can be administered for a period of from one day to six months. For example, the first stage can be administered for one day, two days, three days, four days, five days, six days, seven days, eight days, nine days, ten days, eleven days, twelve days, thirteen days, fourteen days, fifteen days, eighteen days, three weeks, four weeks, five weeks, six weeks, seven weeks, eight weeks, nine weeks, ten weeks, eleven weeks, twelve weeks, thirteen weeks, fourteen weeks, fifteen weeks, four months, five months, or six months.

The third stage of the weaning process may include a composition with comparatively increased amounts of dietary fiber coming from non-milk food than that administered in the first or second stage of the weaning process. For example, the non-milk food can contribute 50% or more of the dietary fiber of the mammal's total daily dietary fiber. If the mammal is an infant that is being weaned while being breast-fed, the mammal may be provided a composition that includes a non-milk food and bifidobacteria, while the mammal is receiving MMOs from another source (e.g., mother's milk). In some embodiments, the composition may include MMO in an amount that is equal to 0-49% of that found in the diet of an exclusively breast-fed infant. In some embodiments, the composition can include MMO in an amount necessary to allow for the total amount of MMO in the mammal to be equal to 0-49% of that found in the diet of an exclusively breast-fed infant.

The third stage of the weaning process can be administered for a period of from one day to six months. For example, the first stage can be administered for one day, two days, three days, four days, five days, six days, seven days, eight days, nine days, ten days, eleven days, twelve days, thirteen days, fourteen days, fifteen days, eighteen days, three weeks, four weeks, five weeks, six weeks, seven weeks, eight weeks, nine weeks, ten weeks, eleven weeks, twelve weeks, thirteen weeks, fourteen weeks, fifteen weeks, four months, five months, or six months.

The overall state of the microbiome at the end of the weaning period should be a range of organisms that cover the breadth of enzymes required to successfully breakdown and sequester all the fermentable fiber in the diet. This a method of building redundancy, so that the genetic capacity held within the foundation of the adult microbiome is equipped to deal with all dietary components a human may encounter. This contributes to the stability.

In various embodiments of the instant invention, the use of the compositions is for the prevention of scours in pre-weaned, weaning, or post weaning pigs, cows, goats, sheep, horses, dogs and cats.

Compositions described above have the listed components combined in ratios and administered in amounts that are effective to accomplish the purposes described for each of the compositions, respectively.

### EXAMPLES

### Example 1. Preparation of a Weaning Food Comprising BMO and its Combination with Activated B. longum Subsp. Infantis

A dry BMO composition is prepared according to Barile⁴ or Christiansen⁵ and comprises about 50% BMO. To a standard commercial baby food recipe, medical food recipe, enteral food, or a food for geriatric patients, is added the BMO preparation at a level of 2 g BMO/oz of commercial food. The MMO composition relative to the food oligosaccharide composition can be determined by GC/MS as in Example 2. A powder composition of activated Bifidobacterium longum subsp. infantis is prepared by a fermentation process known in the art (e.g., as described in PCT/US2015/057226, the contents of which is incorporated herein in its entirety). The final dried B. infantis powder is diluted with infant formula grade lactose to a concentration of 15×10⁹ cfu/g and 0.5 g of the activated Bifidobacterium longum subsp. infantis is added to the commercial food immediately before consumption by a human of any age. ⁴ Barile D, Tao N, Lebrilla C B, Coisson J D, Arlorio M, German J B. (2009) Permeate from cheese whey ultrafiltration is a source of milk oligosaccharides. International Dairy Journal, 19:524-30.⁵ Christiansen, S. et al. (2010) Chemical composition and nutrient profile of low molecular weight fraction of bovine colostrum. International Dairy Journal, 20: p. 630-36

### Example 2. Preparation and Delivery of a Vegetable-Based Weaning Food Composition for a Human Infant that Increases the Microbiome Diversity

Examples of formulation for weaning food for a breast-feeding infant onto complementary foods including probiotics that support the fiber formulation.

A. Pea-Based Weaning Food

A pea puree for a baby is prepared by steaming or boiling peas in a little water for 3-5 minutes and then pureeing the peas with a little of the cooking water using a food processor. The pea puree is then passed through a fine mesh strainer to remove any unpureed bits. Alternatively, a commercial pea-based baby food can be used for the final composition.

A powder composition of Bifidobacterium longum is prepared by fermentation using processes known in the art, such as Kiviharju et al⁶. Glucose, yeast extract and 1-cysteine are used for the cultivation of this strain. Fermentation is carried out at 40 degrees C., in a medium containing 35 g/L yeast extract and 20 g/L glucose. Cultivation is done under anaerobic conditions and the harvested cell suspension is freeze dried according to Kiviharju et al. The final dried powder is diluted with infant formula grade lactose to a concentration of 15×10⁹ cfu/g. ⁶ Kiviharju K, Leisola M, and Eerikäinen T. (2005) Optimization of a Bifidobacterium longum production process. J Biotechnol. 25; 117(3):299-308.

A powder composition of Lactobacillus crispatus is prepared by fermentation using processes known in the art such as Chung et al⁷. A culture of L. crispatus is obtained from a culture collection such as ATCC and is propagated in a fermentation medium comprising 20 g/L glucose, 10 g/L proteose peptone No. 3 (Difco Lab.), 10 g/L beef extract (Difco Lab.), 5 g/L yeast extract (Difco Lab.), 2 g/L ammonium citrate dibasic, 5 g/L sodium acetate trihydrate, 2 g/L dipotassium phosphate, plus micronutrients and antifoam. The fermentation is undertaken in a stirred tank fermentor at 37 C, with agitation at 150 rpm while maintaining a constant pH of 5.5 with acid and/or base additions. The gas phase of the fermentation is maintained anaerobic by using continuously supplied N₂. The harvested cell suspension is freeze dried according to Kiviharju et al. and the final dried powder is diluted with infant formula grade lactose to a concentration of 15×10⁹ cfu/g. ⁷ Chung et al, “Cultivation of Lactobacillus crispatus KLB46 Isolated from Human Vagina,” Biotechnol. Bioprocess Eng. (2001), Vol. 6, pp. 128-132.

Powder compositions comprising B. longum (15×10⁹ cfu/g) and L crispatus (15×10⁹ cfu/g) are blended at a ratio of 1:1 to form a probiotic mixture, and 1 g of the mixture is added as a daily dose to the pea puree immediately before feeding to an infant. Optionally, the probiotic mixture can be provided to the baby one or two days in advance of introduction of the pea-based weaning food.

B. HMO Sweet Potato-Based Weaning Food.

A sweet potato puree was prepared by roasting the sweet potato puree with added water until a smooth texture was reached. An HMO enriched powder, BMO enriched or isolated structures were stirred in to provide a source of MMO. At the time of consumption a sachet containing the probiotic was added before preparation was fed to the infant.

| Ingredient | | New FSM introduced |
|---|---|---|
| HMO enriched powder | 500 mg HMO | none |
| 25 grams sweet potato | 588 mg dietary fiber | Xylose, fructose, GalA |
| B. infantis | 8 B CFU/gram | Galactose, glucose, |
| B. longum | 18 BCFU/gram | Xylose and fructose |
| | | consumer |

C. HMO Green Bean-Based Weaning Food

| Ingredients | | New FSM introduced |
|---|---|---|
| 25 grams green bean puree | 750 mg dietary fiber | Arabinose, Gal A, |
| | | Rhamanose |
| HMO enriched powder | 500 mg HMO | none |
| 25 grams sweet potato | 588 mg dietary fiber | Xylose, fructose, GalA |
| L. reuteri | 18 B CFU | Rhamanose, arabinose |
| | | consumer |
| B. longum | 18 B CFU | |
| B. infantis | 8 B CFU | |

D. BMO Sweet Potato-Based Weaning Food

| Ingredient | | New FSM introduced |
|---|---|---|
| BMO enriched powder | 1 g BMO | none |
| 25 grams sweet potato | 588 mg dietary fiber | Xylose, fructose, GalA |
| 2 FL | 100 mg | None |
| B. infantis | 8 B CFU/gram | Galactose, glucose, |
| B. longum | 18 BCFU/gram | Xylose and fructose |
| | | consumer |

The above formulations were measured for the total potential available free sugar monomer pool by predigesting the dietary fiber prior to analysis. Changing the proportion of the potential FSM relative to the bacterial cultures facilitates development of expansion of the microbiome from infant low diversity to infant higher diversity. The food preparation was separated into an HMO pool and plant oligosaccharide pool from plant polysaccharide by precipitating plant polysaccharide with ethanol. The HMO and plant oligosaccharide were cleaned up with porous graphitized carbon, and injecting HMO and plant polysaccharide fraction into LC-MS instrument for analysis. Polysaccharide were treated with hard acid hydrolysis. Monosaccharide composition was analyzed by permethylation and GC-MS.

### Example 3. Preparation and Delivery of a Cereal-Based Weaning Food for a Human Infant that Increases the Microbiome Diversity

A rice-, oat-, or wheat-based cereals are excellent sources of iron and vitamins. Although there is generally little fiber in rice cereal, cereals containing wheat and oats can be an excellent source of dietary fiber with levels of 2-3 g/serving. Because dietary fiber has a major effect on the microbiome, it is important to match the specific dietary fiber to specific probiotics that can aid in the prevention of excessive availability of FSMs than can lead to pathogenic blooms of bacteria in the baby's gut.

A powder composition of Bifidobacterium bifidum is prepared by fermentation process similar to that of Example 2 for B. longum, and a powder composition of Lactobacillus casei is prepared by fermentation processes similar to that in example 2 for L. crispatus. The final dried powders for both organisms are diluted with infant formula grade lactose to concentrations of 15×10⁹ cfu/g and they are blended at a 1:1 ratio providing a final concentration of 7.5 billion cfu/g of each species. One gram of the probiotic mixture is added as a daily dose to a wheat-based cereal composition immediately before feeding to an infant.

### Example 4. Preparation and Delivery of a Meat-Based Weaning Food for a Human Infant that Increases the Microbiome Diversity

Meat and eggs are indeed perfect weaning foods for a baby. Not only are these animal foods extremely easy to digest compared with cereal grains, but they also supply iron right at the time when a baby's iron stores from birth start to run low, and they are very rich in protein. A chicken puree is prepared by first chopping 1 cup cold and cooked boneless chicken into small 1 inch pieces and placing them in food processor. The food processor is set to puree and the chicken is minced to a powdery mix. The cooking water is added slowly and the mixture is pureed further until a smooth consistency is created. Alternatively, a jar of commercially prepared chicken puree baby food can be used.

A powder composition of Bifidobacterium breve is prepared by fermentation process similar to that of Example 2 for B. longum, and a powder composition of Lactobacillus plantarum is prepared by fermentation processes similar to that in example 2 for L. crispatus. The final dried powders for both organisms are diluted with infant formula grade lactose to concentrations of 15×10⁹ cfu/g and they are blended in a 1:1 ratio providing a final concentration of 7.5 billion cfu/g of each species. One gram of the probiotic mixture is added as a daily dose to a chicken-based infant food composition immediately before feeding to an infant.

### Example 5. Preparation and Delivery of an Antibiotic-Weaning Food for an Adult Human that Increases the Microbiome Diversity

For a regimen of gradual, successive, and gentle increase in microbiome complexity in a patient that is undergoing or has just completed a course of antibiotics, the patient consumes a program of certain combinations of foods and probiotics that may or may not vary in composition or concentration over a period of 1-2 weeks in order for the microbiome to regenerate its initial complexity without the potential for the production of bacterial blooms and subsequent medical consequences such as diarrhea. The daily diets include a total caloric intake of from 1,200-1,800 calories per day for an adult, consisting of servings of 1) peas, rice and avocado; 2) meat or fish; 3) apple or banana; and 4) BMO. BMO intake is 7-10 g per day as a powder, blended in with the pureed fruit or provided as a capsule or enough BMO to represent at least 60% of the total daily dietary fiber. In addition to these foods, a probiotic supplement consisting of B. longum subsp. infantis, B. breve, L. salivarius and L. plantarum is also provided on a daily basis at doses of from 1-5 billion cfu/day of each organism, and the bacteria are provided in an enteric-coated tablet or capsule that has a low-pH protective coating. For individuals that cannot swallow tablets or capsules, the dose is doubled and provided by a powder in a sachet which can be combined with the daily food intake.

### Example 6. Preparation and Use of Therapeutic Compositions for the Treatment of Digestive Pathologies

Bifidobacterium longum subsp infantis was isolated and purified from the feces of a vaginally delivered, breast fed human infant, and its identification was confirmed by DNA analysis that reflected the presence of a gene set that is specifically associated with this organism (Sela et al., 2008, PNAS, 105:18964-18969). A seed culture of this organism was added to a standard growth medium comprising glucose and bovine colostrum as carbon sources in a 500 L agitated fermenter. Following 3 days of growth under anaerobic conditions, a sample of the culture was tested for the presence of activated Bifidobacterium longum subsp. infantis. Activated B. infantis was identified by the presence of gene transcripts for sialidase. The fermenter was harvested by centrifugation, the concentrated cell mass was mixed with a cryopreservative (trehalose plus milk proteins) and freeze dried. The final dry product was 5.5 kg of bacterial mass with a live cell count of 1.30×10¹¹ cfu/g.

The activated B. infantis product was blended with pharmaceutical grade lactose to provide a minimum dose of 30 Billion cfu of B. longum subsp. infantis per gram. 0.625 g of this diluted activated B. infantis product was then packaged in oxygen- and moisture-resistant sachets, to provide doses of 15 Billion cfu of B. longum subsp. infantis per sachet. One sachet of 18 billion cfu of B. longum subsp. infantis was consumed with a morning breakfast and one with an evening meal.

A concentrated mixture of bovine milk oligosaccharide (BMO) was obtained from whole milk which was pasteurized by heating to 145 degrees F. for 30 minutes, cooled and centrifugally defatted, separating it into cream (predominantly fat) and skim milk (defatted product). The defatted skim milk was then ultra-filtered using membranes with a 5-10 kDa cut off to concentrate a protein fraction (predominantly whey, proteins and caseins). The lactose in the permeate was partially eliminated by an additional nanofiltration using a 1 kDa cut off. The composition was then spray dried. This composition of dried BMOs comprised about 15% lactose and about 10% BMO with the remainder of the mass primarily peptides, ash and other components. Twenty grams of this BMO composition was combined with 5 g of GOS (Vivinal GOS) as the daily ration for treatment.

The BMO preparation was packaged in separate bags and administered in a daily ration of 20 g BMO+5 g GOS. Each of the bags of BMO provided specific energy support for the growth of the organism (B. longum subsp. infantis) in the colon of the patient, which thereby provided a gut environment favoring mucosal healing.

The use of the therapeutic composition providing both the activated B. infantis and the source of MMO (i.e. BMO) required a substantive change in the adult diet. The dietary fiber source needed to be switched from a predominantly plant-based adult diet to a predominantly milk based infant diet. This required the adult to follow a new regime to make this transition. The new diet regime provided essentially no non-milk fiber and replaced it with the milk fiber. The BMO was consumed 5 times per day (5×4 g of the BMO powder of Example 2), approximately every 3-4 hr. by blending the 4 g of powder with a meal replacer (Boost, Nestle Nutrition) containing 240 Cal/drink with 15 g/protein and 6 g of fat and 0 g of dietary fiber. The patient was allowed to consume 2-3 eggs each morning, and one serving of fish or meat with lunch and dinner. Any dietary fiber consumption other than the BMO was kept at less than 1 g/day.

As a step to accelerate the switch from a microbiome consuming adult dietary fiber to a microbiome consuming milk-based fiber, the subjects completed a colonoscopy preparation involving a clear liquid diet and laxatives to clear out the bowels of fiber and temporarily reducing or destabilizing the microbial biomass in preparation for the diet change. Once this was completed, the subject followed the specific diet that limited non-milk based fiber to less than 1 gram per day and ensured the subject was eating a diet with sufficient protein, fat and carbohydrate to maintain a healthy weight.

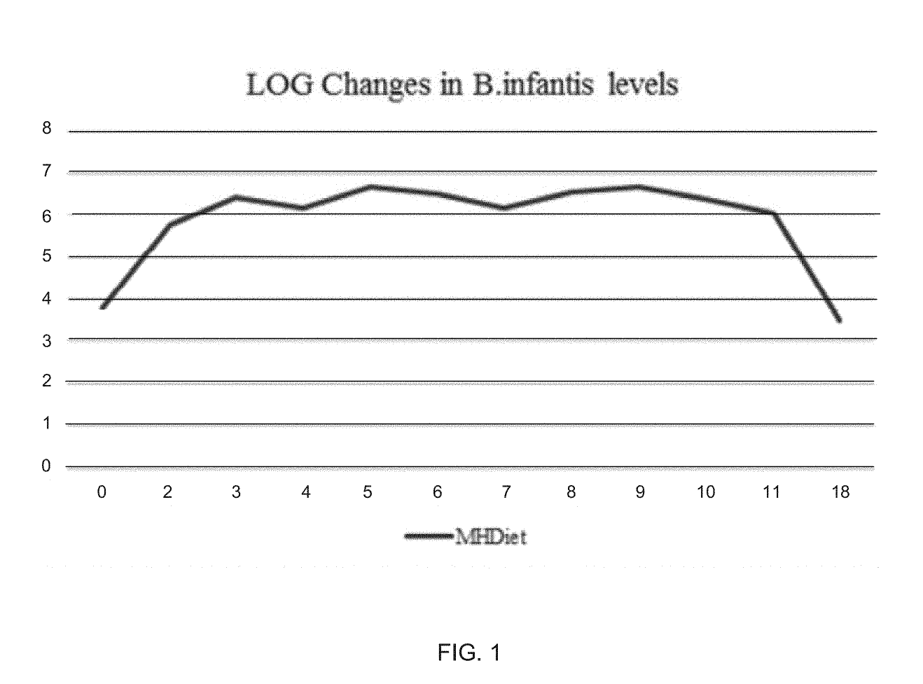

Fecal samples were taken the day before the colonoscopy prep (pretreatment) and on a daily basis for the 7 days on the dietary regiment of consumption of the B. infantis and BMO. The subject also filled out questionnaire forms regarding a self-assessment of his gastrointestinal responses or indicators of the palliative effect of the composition on symptoms of gastrointestinal distress. Following the seven days of dietary regimen, the subject patient was allowed to return to his pretreatment standard diet and post treatment fecal samples were taken during a 1 week post-treatment phase. DNA was extracted and subjected to qPCR analysis and NextGen sequencing for microbiome analysis. B. infantis was specifically measured using qPCR (FIG. 1). At baseline, B. infantis was below the limit of detection in an adult gut. Detectable levels were observed with supplementation and diet changes. As shown in FIG. 1, there was a 3 LOG difference between baseline and during treatment. The NGS data provided a means of visualizing the relative changes in different clades and families of bacteria. Samples were also prepared for other measurements including BMO content by Mass Spectrometry in the stool to monitor in vivo consumption, short chain fatty acid and lactate, pH determinations, measurements of cytokines and a full metabolomics determination.

TABLE 1
Study Schedule
BMO/GOS Treated Participant

| | D 0 | D 1 | D 2 | D 3 | D 4 | D 5 | D 6 | D 7 | D 8 | D 9 | D 10 | D 11 | D 18 |
|---|---|---|---|---|---|---|---|---|---|---|---|---|---|
| Colonoscopy Prep | | X | | | | | | | | | | | |
| Regular diet | X | | | | | | | | | X | X | X | X |
| No fiber diet | | X | X | X | X | X | X | X | X | | | | |
| BMO & GOS | | X | X | X | X | X | X | X | X | | | | |
| B. infantis (1AM & | | | X | X | X | X | X | X | X | | | | |
| 1PM) | | | | | | | | | | | | | |
| Swab (x2) | X | | X | X | X | X | X | X | X | X | X | X | X |
| Stool | X | | X | X | X | X | X | X | X | X | X | X | |

### Example 7. Colonic Mucosal Preparation Prior to a FMT that Increases the Microbiome Diversity

Prior to a fecal microbial transplant, the patient undergoes a colonic mucosal preparation regimen consisting of a dietary preparation period of 5 days wherein the patient consumes a total of 10 g/d of the BMO powder of Example 1 blended in whole or in part with and/or consumed contemporaneously with the patient's daily meals, where the BMO represents at least 70% of the total daily dietary fiber consumed by the patient. During the 5-day preparation period, the patient will also, preferably consume a daily dose of 5×10¹⁰ cfu of Bifidobacterium longum subsp. infantis prepared as in Example 6. Immediately before the fecal transplant, a composition comprising 5×10¹⁰ cfu of Bifidobacterium longum subsp. infantis in 5 g BMO of Example 1 is mixed with the FMT composition and provided directly to the patient as an enema or other device used to deliver the fecal transplant.

### Example 8. Preparation and Delivery of a Vegetable-Based Weaning Food Composition for a Human Infant that Increases the Microbiome Diversity

A pea puree for a baby is prepared by steaming or boiling peas in a little water for 3-5 minutes and then pureeing the peas with a little of the cooking water using a food processor. The pea puree is then passed through a fine mesh strainer to remove any unpureed bits. Alternatively, a commercial pea-based baby food can be used for the final composition. The BMO composition of Example 1 is added to this puree or commercial pea-based baby food in an amount of 0.5 g BMO preparation/oz of baby food.

A powder composition of Bifidobacterium longum is prepared by a process similar to that described in Example 1. The final dried powder is diluted with infant formula grade lactose to a concentration of 15×10⁹ cfu/g.

A powder composition of Lactobacillus crispatus is prepared by a process similar to that described in Example 2. The final dried powder is diluted with infant formula grade lactose to a concentration of 15×10⁹ cfu/g.

Powder compositions comprising B. longum (15×10⁹ cfu/g) and L crispatus (15×10⁹ cfu/g) are blended at a ratio of 1:1 to form a probiotic mixture, and 1 g of the mixture is added as a daily dose to the pea puree/BMO mixture immediately before feeding to an infant. Optionally, the probiotic mixture can be provided to the baby one or two days in advance of introduction of the pea-based weaning food.

### Example 9. Preparation and Delivery of a Cereal-Based Weaning Food for a Human Infant that Increases the Microbiome Diversity

A rice-, oat-, or wheat-based cereals are excellent sources of iron and vitamins. Although there is generally little fiber in rice cereal, cereals containing wheat and oats can be an excellent source of dietary fiber with levels of 2-3 g/serving.

A powder composition of Bifidobacterium bifidum is prepared by a process similar to that of Example 1, and a powder composition of Lactobacillus casei is prepared by a process similar to that in Example 2. The final dried powders for both organisms are diluted with infant formula grade lactose to concentrations of 15×10⁹ cfu/g and they are blended at a 1:1 ratio providing a final concentration of 7.5 billion cfu/g of each species. One gram of the probiotic mixture is added as a daily dose to a wheat-based cereal composition immediately before feeding to an infant.

### Example 10. Preparation and Delivery of a Meat-Based Weaning Food for a Human Infant that Increases the Microbiome Diversity

Meat and eggs are indeed perfect weaning foods for a baby. Not only are these animal foods extremely easy to digest compared with cereal grains, but they also supply iron right at the time when a baby's iron stores from birth start to run low; and they are very rich in protein. A chicken puree is prepared by first chopping 1 cup cold and cooked boneless chicken into small 1 inch pieces and placing them in food processor. The food processor is set to puree and the chicken is minced to a powdery mix. The cooking water is added slowly and the mixture is pureed further until a smooth consistency is created. Alternatively, a jar of commercially prepared chicken puree baby food can be used.

A powder composition of Bifidobacterium breve is prepared by a process similar to that described in Example 1, and a powder composition of Lactobacillus plantarum is prepared by a process similar to that in Example 2. The final dried powders for both organisms are diluted with infant formula grade lactose to concentrations of 15×10⁹ cfu/g and they are blended in a 1:1 ratio providing a final concentration of 7.5 billion cfu/g of each species. One gram of the probiotic mixture is added as a daily dose to a chicken-based infant food composition immediately before feeding to an infant.

### Example 11. Delivery of a Weaning Food Protocol for a Nursing Mammalian Infant that Increases the Microbiome Diversity

A low-diversity microbiome is first established using breast milk supplemented with B. infantis (10×10⁹ cfu/d) prepared according to Example 1 for a period of one week. This first step establishes a B. infantis-dominated microbiome is a starting point for wean but is not necessary if the infant is already exclusively nursing and has a gut microbiome already dominated by B. infantis. After the establishment of the B. infantis-dominant microbiome, the second step (initiation of weaning) begins wherein the infant is given a composition that includes B. infantis and a non-milk food, where the non-milk food contributes from 10% to 49% of the dietary fiber of the mammal's total dietary fiber intake. This second step takes place over a period of three weeks. During this time the infant is also receiving MMO from breast milk, though the amount of MMO from breast milk is less than the infant was being provided in stage one. After the period of three weeks for stage two, the infant is given a composition that includes B. infantis and a non-milk food, where the non-milk food contributes from 50% to 100% of the dietary fiber of the mammal's total dietary fiber intake for a period of three weeks. The infant may also receive MMO from breast milk, though the amount of MMO from breast milk is less than the mammalian infant was being provided in stage one and stage two.

### Example 12. Delivery of a Weaning Food Protocol for a Nursing Mammalian Infant that Increases the Microbiome Diversity

For a period of one week (Stage One), while the mammalian infant is nursing, the infant is introduced to the weaning food composition of Example 8 that includes B. longum, L. crispatus and the pea puree. The amount of the pea puree introduced to the mammalian infant contributes 10% or less of the dietary fiber of the mammal's total daily dietary fiber (MMO plus pea puree) for the first week.

In Stage Two, the daily amount of the weaning food composition of Example 8 provided to the mammalian infant is increased to a level where the pea puree now contributes from 10% to 49% of the dietary fiber of the infant mammal's total daily dietary fiber (MMO plus pea puree) for a period of three weeks. The mammalian infant is receiving MMO from breast milk, though the amount of MMO from breast milk is less than the mammalian infant was being provided in Stage One.

In Stage Three the daily amount of the weaning food composition of Example 8 provided to the mammalian infant is increased to a level where the pea puree now contributes from 50% to 100% of the dietary fiber of the mammal's total dietary fiber (MMO plus pea puree) intake for a period of three weeks. The mammalian infant may also receive MMO from breast milk, though the amount of MMO from breast milk is less than the mammalian infant was being provided in stage one and Stage Two.

### Example 13. Weaning of a Breast-Fed Infant onto Infant Formula

A. Breast Fed Infants

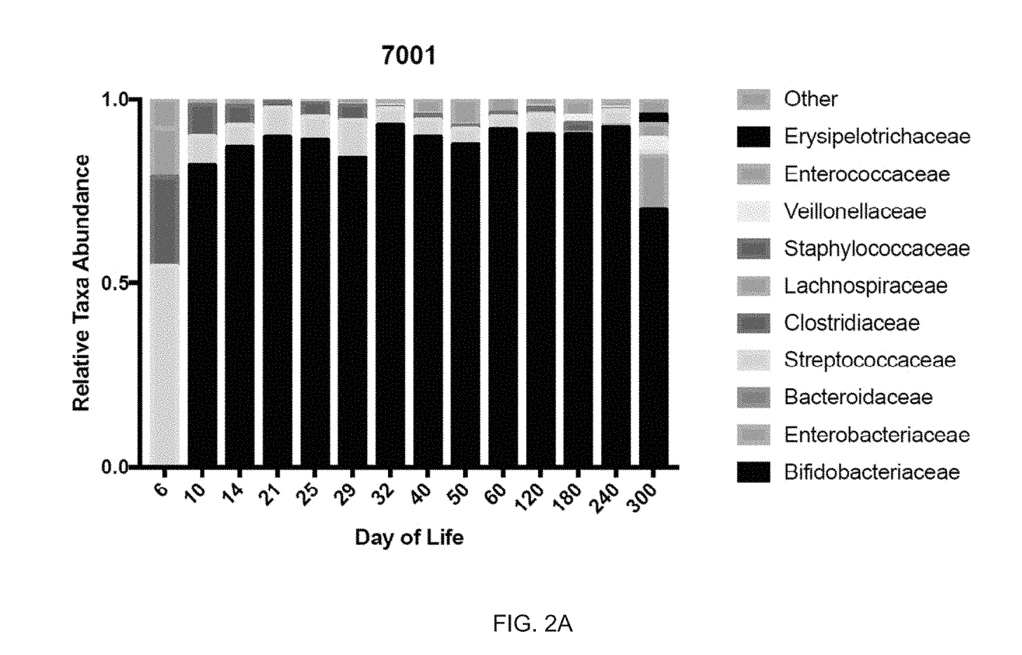

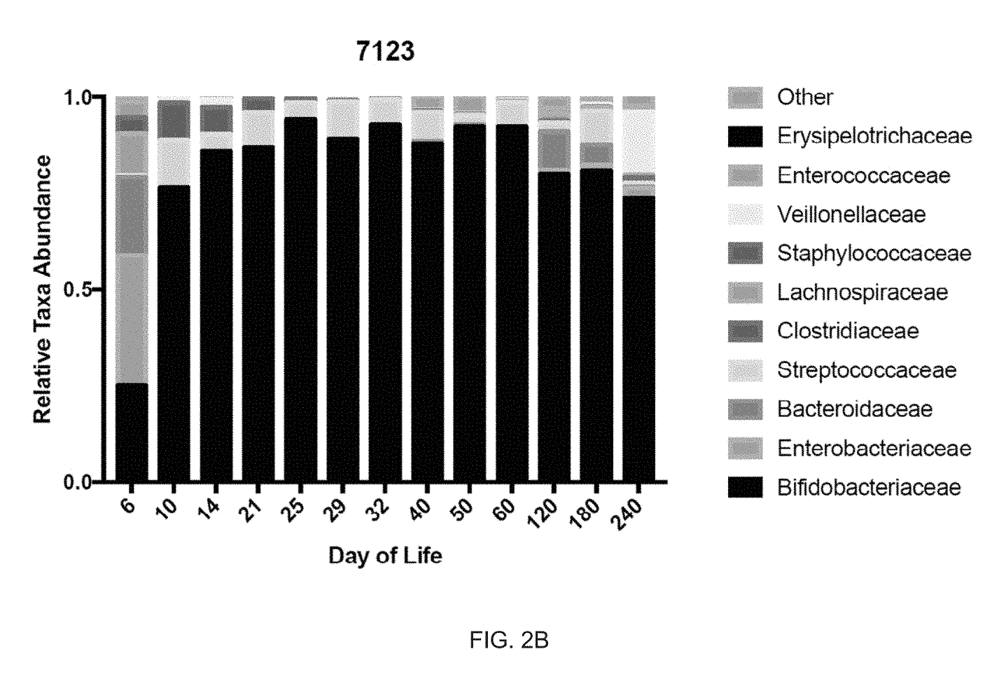

Infants were given 18 billion CFU B. infantis mixed with 5 mLs breast milk in a medicine cup and fed with a feeding syringe from day 7 to day 28 of life. This established a simple microbiome (high Bifidobacterium) that persisted as long as infants were breast feeding (FIGS. 2A and 2B). Infants were followed for 1 year. Recall questionnaires were completed by the mother on any dietary changes including any formula and/or complementary feeding. These subjects were not given formula during the year. It was demonstrated that complementary foods up to 20 tbsp. did not have an appreciable effect on the microbiome during the first year of life when breast feeding is continued (FIGS. 2A & 2B).

B. Weaning to Formula

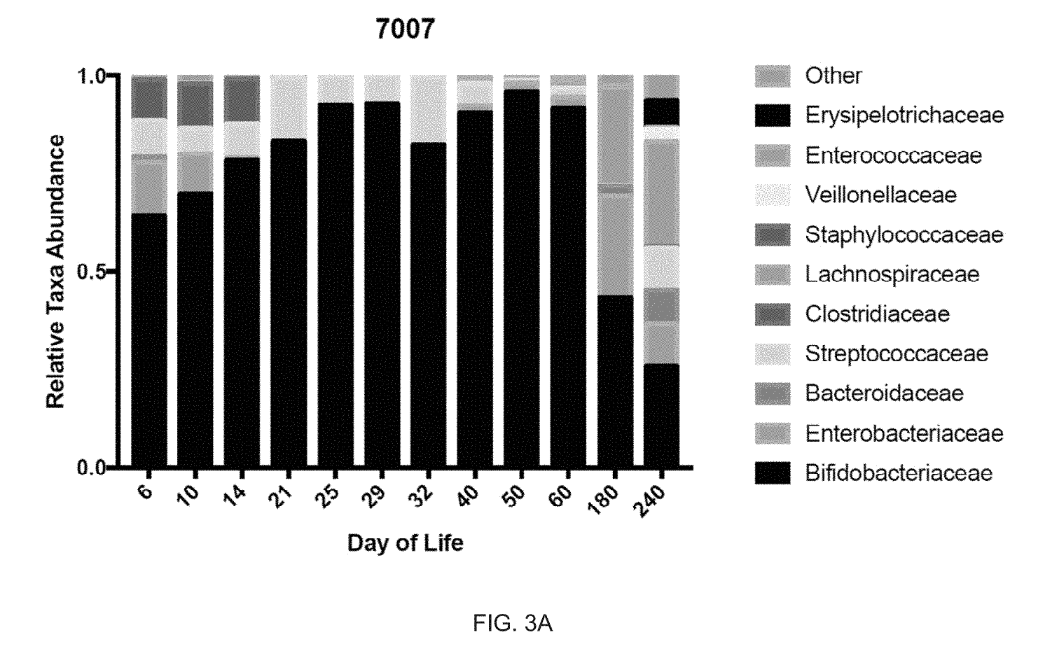

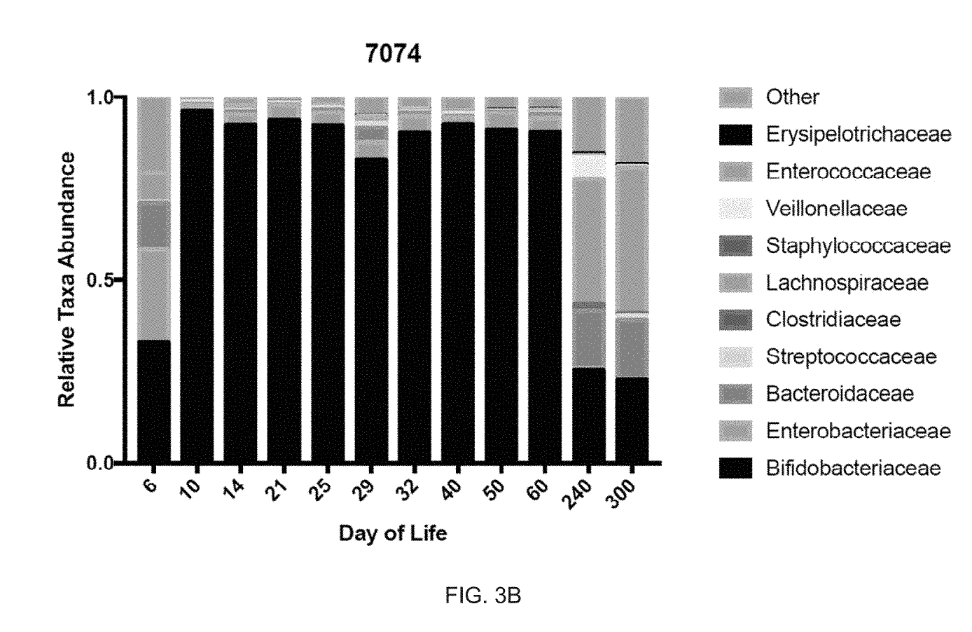

In the first six months of life, an infant whose diet switches from exclusively breast milk to infant formula requires a formulation comprising B. infantis plus MMO to replace 100% of the HMO being lost from the diet. In the case of mixed feeders, the amount of MMO required is dependent on the number of formula bottles that displace an equivalent feeding of breast milk. Infants who switched to formula during the first year of life are represented in FIGS. 3A & 3B.

The following table demonstrates a proposed feeding regime for infants 0-6 months of age:

| Breast Milk Diet | Formula Diet | MMO Required |
|---|---|---|
| 75% | 25% | 1 gram |
| 50% | 50% | 1.9 grams |
| 25% | 75% | 2.8 grams |
| 0% | 100% | 3.75 grams |

The table may be expanded for infants up to 1 year and beyond, by displacing portions of MMO with other dietary fiber.

### Example 14. Weaning of Non-Human Animals

In untreated young nursing pigs, populations of Enterobacteriaceae in the gut were found to correlate with the abundance of Bacteroides (r2=0.661, p<0.001). It was also found that these populations of Enterobacteriaceae cannot, by themselves, consume sialylated pig milk oligosaccharides, but Bacteroides possess enzymes capable of releasing sialic acid from pig milk oligosaccharides, which is associated with increased abundances of sialic acid in feces. Enterobacteriaceae can consume the sialic acid released by Bacteroides. The treatment of pigs with Bifidobacterium and/or Lactobacillus reduced the amount of sialic acid available and the treatment resulted in a reduction in scours (See WO 2016/094836 & WO 2016/149149, the contents of which are incorporated herein in their entirety).

Newborn foals were treated with a probiotic combination of B. infantis and Lactobacillus plantarum twice a day for 4 days while nursing (which provided a source of mare's milk oligosaccharides). The effect on foal heat diarrhea (weaning induced diarrhea and GI distress) was studied. This probiotic preparation reduced foal heat diarrhea in 100% of treated animals compared to animals not receiving the probiotic product. See U.S. Patent No. 62/307,420, the contents of which is incorporated herein in its entirety.

### REFERENCES

‘Blooming’ in the gut: how dysbiosis might contribute to pathogen evolution
Barbel Stecher, Lisa Maier & Wolf-Dietrich Hardt
Nature Reviews Microbiology 11, 277-284 (April 2013)

TABLE 1
Listing of common weaning foods and expected Free Sugar
Monomers to be released under normal digestive processes.

| | Monosaccharides | | | | | | | | | | | | |
|---|---|---|---|---|---|---|---|---|---|---|---|---|---|
| Weaning Foods | Glc | Gal | Man | Xyl | Fru | Rha | Neu5Ac | Neu5Gc | GlcNAc | GalNAc | Arb | GlcA | GalA |
| Grains/Cereals | | | | | | | | | | | | | |
| Barley | x | | x | x | | | | | | | x | | |
| Corn | | | | | | | | | | | | | |
| hummus | | | | | | | | | | | | | |
| Lentils | x | x | | | x | | | | | | | | |
| Oats | | | | | | | | | | | | | |
| Rice | x | | | | | | | | | | | | |
| Wheat | X | | | x | X | | | | | | x | | |
| Vegetables | | | | | | | | | | | | | |
| Avocado | X | | | | X | | | | | | | | |
| Beetroot | X | | | | X | | | | | | | | |
| Broccoli | X | | | | X | | | | | | | | |
| Squash | X | | | X | | | | | | | X | X | X |
| Carrots | X | X | | X | X | | | | | | | X | X |
| Green Beans | | X | | | | X | | | | | X | | X |
| Peas | X | | | | X | | | | | | | | |
| Potato | X | X | | X | X | X | | | | | | | X |
| Casava | X | X | | X | X | X | | | | | | | X |
| Sweet potato | X | X | | X | X | | | | | | | | X |
| Pumpkin | X | X | | | | | | | | | | | |
| Fruits | | | | | | | | | | | | | |
| Apple | X | X | | X | | | X | | | | X | | X |
| banana/plantain | X | X | | X | | | X | | | | X | | X |
| Blueberry | X | X | | X | | | X | | | | X | | X |
| Mango | X | X | | X | | | X | | | | X | | X |
| Peach | X | X | | X | | | X | | | | X | | X |
| Pear | X | X | | X | | | X | | | | X | | X |
| Papaya | X | X | | X | | | X | | | | X | | X |
| Watermelon | X | X | | X | | | X | | | | X | | X |
| Meats | X | X | X | | | | X | | X | X | | | |
| Fish | X | X | X | | | | X | | X | X | | | |
| Cheese/Dairy | X | X | X | | | | X | X | X | X | | | |

TABLE 2
Listing of common intestinal microbiota and preferences for free sugar consumption.

| | | | | | | | | |
|---|---|---|---|---|---|---|---|---|
| | Sialic | | | | | | | |
| Fucose | Acid | N-Acetylglucosamine | Glucose | Galactose | Lactose | Sialyllactose | Fucosyllactose | Lacto-N-Biose |

Bifidobacterium

| | | | | | | | | | |
|---|---|---|---|---|---|---|---|---|---|
| B. infantis | * | + | + | + | + | + | + | + | + |
| B. breve | * | + | + | + | + | + | + | + | + |
| B. bifidum | * | − | + | + | + | + | + | + | + |
| B. longum | * | − | + | + | + | + | − | − | + |
| B. adolescentis | − | − | + | + | + | + | − | − | − |
| B. animalis | − | − | − | + | + | + | − | − | − |

Lactobacillus

| | | | | | | | | | |
|---|---|---|---|---|---|---|---|---|---|
| L. reuteri | − | − | + | + | + | + | − | − | − |
| L. acidophilus | − | − | + | + | + | + | − | − | − |
| L. plantarum | − | + | + | + | + | + | − | − | − |
| L. casei | − | − | + | + | + | + | − | + | + |
| L. rhamnosus | − | − | + | + | + | + | − | + | − |
| L. brevis | − | − | + | + | + | + | + | − | − |
| L. fermentum | − | − | − | + | + | + | − | − | − |
| L. crispatus | − | + | + | + | + | + | − | − | − |
| L. johnsonii | − | − | + | + | + | + | − | − | − |
| L. gasseri | − | + | + | + | + | + | − | − | − |
| L. mucosae | − | − | − | + | + | + | − | − | − |
| L. salivarius | − | + | + | + | + | + | + | − | − |

Pediococcus

| | | | | | | | | | |
|---|---|---|---|---|---|---|---|---|---|
| P. stilesii | + | ND | + | + | + | + | ND | ND | ND |
| P. pentosaceus | + | ND | + | + | + | + | ND | ND | ND |
| P. acidilacti | − | ND | + | + | + | − | ND | ND | ND |
| P. argentinicus | − | ND | + | + | + | − | ND | ND | ND |

| | | | | | | |
|---|---|---|---|---|---|---|
| Mannose | Xylose | Fructose | Rhamnose | Arabinose | Glucuronate | Galacturonate |

Bifidobacterium

| | | | | | | | |
|---|---|---|---|---|---|---|---|
| B. infantis | −/v | − | + | − | − | + | − |
| B. breve | + | − | + | − | − | − | − |
| B. bifidum | − | − | + | − | − | − | − |
| B. longum | − | + | + | − | + | − | − |
| B. adolescentis | − | − | + | − | − | − | − |
| B. animalis | − | + | − | − | + | − | − |

Lactobacillus

| | | | | | | | |
|---|---|---|---|---|---|---|---|
| L. reuteri | + | − | + | + | + | ND | ND |
| L. acidophilus | + | − | + | − | − | ND | ND |
| L. plantarum | + | − | + | − | + | ND | ND |
| L. casei | + | − | + | − | − | ND | ND |
| L. rhamnosus | + | − | + | + | − | ND | ND |
| L. brevis | − | v | + | − | + | ND | ND |
| L. fermentum | + | − | + | − | v | ND | ND |
| L. crispatus | + | − | + | − | − | ND | ND |
| L. johnsonii | + | − | + | − | − | ND | ND |
| L. gasseri | + | − | + | − | − | ND | ND |
| L. mucosae | − | + | + | − | + | ND | ND |
| L. salivarius | + | − | + | + | − | ND | ND |

Pediococcus

| | | | | | | | |
|---|---|---|---|---|---|---|---|
| P. stilesii | + | − | + | + | − | ND | ND |
| P. pentosaceus | + | − | + | − | + | ND | ND |
| P. acidilacti | + | + | + | − | + | ND | ND |
| P. argentinicus | + | − | + | − | − | ND | ND |

* = predicted, but not observed;
ND = Not Determined

1. A composition comprising a non-milk food and a bacterial culture.

2. The composition of claim 1, wherein the bacterial culture comprises commensal bacteria.

3. The composition of claim 1 or 2, wherein the bacterial culture is selected from the species of Lactobacillus, Bifidobacterium, Pediococcus and combinations thereof.

4. The composition of any one of claims 1-3, further comprising mammalian milk oligosaccharides (MMO).

5. The composition of claim 4, wherein the MMO is from a human, bovine, equine, or caprine source.

6. The composition of claim 4 or 5, wherein the MMO comprises HMO, BMO, BCO, GMO, GOS, or combinations thereof.

7. The composition of any one of claims 4-6, wherein the dietary fiber in the composition comprises at least 20% of MMO.

8. The composition of any one of claims 4-7, wherein the dietary fiber in the composition comprises at least 30% of MMO.

9. The composition of any one of claims 4-8, wherein the dietary fiber in the composition comprises at least 40% of MMO.

10. The composition of any one of claims 4-9, wherein the dietary fiber in the composition comprises at least 50% of MMO.

11. The composition of any one of claims 4-10, wherein the dietary fiber in the composition comprises at least 60% of MMO.

12. The composition of any one of claims 4-11, wherein the dietary fiber in the composition comprises at least 70% of MMO.

13. The composition of any one of claims 1-12, wherein less than 10% of the dietary fiber in the composition is provided by the non-milk food.

14. The composition of any one of claims 1-13, wherein the bacterial culture is provided in a dose from 10⁷-10¹² cfu.

15. The composition of any one of claims 1-14, wherein the non-milk food is a feed for a non-human mammal.

16. The composition of claim 15, wherein the non-human mammal is a pig, cow, goat, sheep, horse, dog, or cat.

17. The composition of any one of claims 1-14, wherein the non-milk food is a food for a human.

18. The composition of claim 17, wherein said human is a baby.

19. The composition of any one of claim 17 or 18, wherein the human has a microbiome in need of increasing its complexity by at least 10%.

20. The composition of any one of claims 17-19, wherein the human has a microbiome in need of increasing its complexity by at least 20%.

21. The composition of any one of claims 17-20, wherein the human has a microbiome in need of increasing its complexity by at least 30%.

22. The composition of any one of claims 17-21, wherein the human is receiving or has just completed a course of oral antibiotics.

23. The composition of any one of claims 17-22, wherein the human is receiving or has just completed a course of chemotherapy.

24. The composition of any one of claims 17-23, wherein the human is receiving or has just completed a fecal microbial transplant.

25. The composition of any one of claims 3-24, wherein the Bifidobacteria is selected from B. longum subsp longum, B. longum subsp infantis, B. breve, Bacterium pseudocatanulatum, B. bifidum, B. adolescentis, B. pseudolongum, and B. animalis.

26. The composition of any one of claims 3-25, wherein the Lactobacillus is selected from L. crispatus, L. casei, L. silivarius, L. reuteri, and L. plantarum.

27. The composition of any one of claims 3-26, wherein the Pediococcus is selected from P. pentosaceus, P. stilesii, P. acidilacti, P. argentenicus, and P. claussenii.

28. The composition of any one of claims 1-27, wherein the non-milk food comprises complex oligosaccharides from meat, fish, eggs, shellfish, fruits, vegetables, grains, nuts, seeds, or combinations thereof.

29. The composition of claim 28, wherein the non-milk food comprises complex oligosaccharides from barley, corn, hummus, lentils, oats, rice, wheat, avocado, beetroot, broccoli, squash, carrots, green beans, peas, potatoes, cassava, sweet potatoes, pumpkin, apples, bananas, plantains, blueberries, mango, peach, pear, papaya, watermelon, or combinations thereof.

30. A composition of a non-milk food comprising MMO from a human, bovine or caprine source.

31. A composition of a non-milk food comprising HMO, BMO, BCO, GMO, GOS, or combinations thereof.

32. The composition of claim 30 or 31, further comprising and a bacterial culture.

33. The composition of claim 32, wherein the bacterial culture comprises bacteria of the genus Bifidobacteria, Lactobacillus, Pediococcus, or combinations thereof.

34. The composition of any one of claims 30-33, wherein the non-milk food is a food for a human baby.

35. A method of increasing the gut microbiome complexity in a subject in need thereof comprising administering to the subject in need thereof a composition of any one of claims 1-33.

36. The method of claim 35, wherein the subject in need thereof is receiving antibiotic therapy.

37. The method of claim 35, wherein the subject in need thereof has recently finished antibiotic therapy.

38. The method of any one of claims 35-37, wherein the subject in need thereof is receiving or has recently received a fecal microbial transplant.

39. The method of any one of claims 35-38, wherein the subject in need thereof is a human.

40. The method of claim 39, wherein the human is not an infant.

41. The method of any one of claim 39 or 40, wherein the human is an adult.

42. A method of increasing the gut microbiome complexity in a subject in need thereof comprising administering a non-milk food, MMO, and a bacterial culture to the subject in need thereof.

43. The method of claim 42, wherein the MMO are derived from or substantially identical to human milk glycans, bovine milk glycans, or goat milk glycans.

44. The method of claim 42, wherein the MMO comprises HMO, BMO, BCO, GMO, GOS, or combinations thereof.

45. The method of any one of claims 42-44, wherein the bacterial culture comprises bacteria from Bifidobacteria, Lactobacillus, Pediococcus, or combinations thereof.

46. The method of claim 45, wherein the bacterial culture comprises B. infantis, B. breve, L. plantarium, L. reuteri, or combinations thereof.

47. The method of claim 46, wherein the bacterial culture is provided in a daily dose of from 10⁷-10¹² cfu.

48. The method of any one of claims 42-47, wherein the milk glycans are added to the food such that they represent at least 20% of the total daily dietary fiber of the diet.

49. The method of any one of claims 42-48, wherein the bacterial culture comprises Bifidobacteria longum subsp. infantis.

50. The method of any one of claims 42-49, wherein the subject in need thereof is receiving antibiotic therapy.

51. The method of claim 50, wherein the subject in need thereof has recently finished antibiotic therapy.

52. The method of any one of claims 42-51, wherein the subject in need thereof is receiving or has recently received a fecal microbial transplant.

53. The method of any one of claims 42-52, wherein the subject in need thereof is a human.

54. The method of claim 53, wherein the human is an adult.

55. A method of increasing the gut microbiome complexity in a subject in need thereof comprising administering a fecal microbial transplant composition comprising a fecal microbiome from a healthy individual, milk glycan, and a bacterial culture.

56. The method of claim 55, wherein the fecal microbial transplant composition comprises 5 g of the milk glycan.

57. The method of any one of claim 55 or 56, wherein the bacterial culture comprises bacteria of the genus Bifidobacteria, Lactobacillus, Pediococcus, or combinations thereof.

58. The method of any one of claims 55-57, wherein the bacterial culture comprises Bifidobacterium infantis subsp infantis.

59. The method of any one of claims 56-58, wherein the bacterial culture comprises from 1×10⁶ to 100×10⁹ cfu of the bacteria.

60. The method of any one of claims 55-59, wherein the subject in need thereof has been given a controlled diet prior to the fecal microbial transplant.

61. The method of claim 60, wherein the controlled diet comprises low-fiber food and milk glycan

62. The method of claim 61, wherein the controlled diet comprises 1-30 g per day of the milk glycan.

63. The method of any one of claims 60-62, wherein the controlled diet has been administered to said subject for a period of from at least 1 to 7 days prior to the fecal microbial transplant.

64. The method of claim 63, wherein the milk glycan comprises from at least 20% to at least 70% of the total dietary glycans of the controlled diet.

65. A method of increasing the gut microbiome complexity in a human in need thereof comprising administering a composition, wherein the composition comprises weaning food and a bacterial culture, and wherein the composition is in a dry powder form.

66. The method of claim 65, wherein the bacterial culture comprises bacteria from the genus Bifidobacterium, Lactobacillus, or combinations thereof.

67. The method of claim 61, wherein the bacterial culture comprises B. infantis, B. breve, L. planatrum, L. reuteri, or combinations thereof.

68. The method of any one of claim 65 or 66, wherein the bacterial culture further comprises a preservative.

69. The method of any one of claims 65-68, wherein the weaning food comprises meat, fish, eggs, shellfish, fruits, vegetables, grains, nuts, seeds, dairy, or combinations thereof.

70. The composition of claim 69, wherein the weaning food comprises barley, corn, hummus, lentils, oats, rice, wheat, avocado, beetroot, broccoli, squash, carrots, green beans, peas, potatoes, cassava, sweet potatoes, pumpkin, apples, bananas, plantains, blueberries, mango, peach, pear, papaya, watermelon, cheese, or combinations thereof.

71. The method of any one of claims 65-70, wherein the composition comprises bacterial culture and weaning food in a ratio of from 10⁶-10¹¹ cfu of bacterial culture to 100 g of weaning food.
